(19) United States
(12) Patent Application Publication
(10) Pub. No.: US 2003/0054020 A1
(43) Pub. Date: Mar. 20, 2003

(54) Method and composition for reducing sebum secretion in mammals

(76) Inventor: Sarfaraz Niazi, Chicago, IL (US)

Correspondence Address: Gerald T. Shekleton, Esq., Welsh & Katz, Ltd., 22nd Floor, 120 S. Riverside Plaza, Chicago, IL, 60606, US

(21) Appl. No.: 09/949,445

(22) Filed: Sep. 7, 2001

(51) Int. Cl. A61K 6/00; A61K 7/00

(52) U.S. Cl. 424/401

(57) ABSTRACT

This invention relates to reducing sebum production on the skin using methods and compositions containing a surfactant, a chylomicron disrupter, a skin penetration enhancer, and an anti-androgenic compound. In a preferred embodiment, the composition contains the surfactant as a mixture of polyoxyethylene compounds and the anti-androgenic agent as a mixture of saw palmetto extract and nettle extract.

### TECHNICAL FIELD

This invention relates to cosmetic compositions and methods for reducing oily skin conditions in mammals by topical application of compositions containing a carrier, surfactants, chylomicron disrupters, skin penetration enhancers, and anti-androgenic compounds.

### BACKGROUND OF THE INVENTION

Cosmetic products which improve the condition and appearance of skin are in great demand. Perhaps the most prevalent skin condition for which remedy is sought is excessive skin oiliness, particularly in the facial area. Excessive oiliness results from large amounts of sebum being secreted onto the skin surface. Sebum is a complex fatty mixture which is produced by cells of the sebaceous glands in the skin (sebocytes). Once produced, the sebum usually is secreted up hair follicles to the skin surface. Sometimes the secretion process is blocked and can lead to disorders such as acne.

The primary lesion of acne is the comedo. The open comedo (blackhead) consists of a firm mass of keratin and sebum, which blocks and dilates the follicle pore. The upper portion of the blackhead is darkened by slow oxidative changes (not by dirt), and the lower portions are white. The closed comedo (whitehead), which is a collection of skin cells and sebum with the hair follicular opening blocked, are potentially the starting point of deep inflammatory lesions.

There are many remedies available to treat the symptoms of oily skin. Cleansing agents such as abrasives, astringents, special soaps, etc. have been widely used, however these merely reduce or remove surface lipids temporarily (see for example U.S. Pat. No. 4,588,750 to Boris, U.S. Pat. No. 6,019,975 to Bajor et al., and U.S. Pat. No. 5,690,948 to McCook et al.). Topical drying agents such as sulfur, resorcinol, and salicylic acid have also been used but their mode of action produces erythema (reddening) and desquamation (peeling) of the outer surface of the skin which is intolerable and undesirable to many consumers.

Another common remedy for oily skin is the use of antibiotics. However, side effects are very common in this method as well, the most prominent being the development of resistant bacterial organisms. Sensitization of the skin is also prevalent with antibiotic therapy.

Recently, it has been demonstrated that compounds which reduce sebum production can lessen the severity of acne. Examples of such compounds are 13-cis-retinoic acid, spironolactone, and cyproterone acetate (see U.S. Pat. No. 4,367,227 to Bingham). It has been shown that 13-cis-retinoic acid can reduce the size of human sebaceous glands by up to 90 percent. However, this compound can sometimes cause serious side effects such as hypervitaminosis A, a form of vitamin A poisoning, so its use is very limited.

An alternative way to reduce sebum production is to reduce the cellular response and therefore the production of sebum. This cellular response in humans is partially controlled by the androgenic hormone systems. Anti-androgenic agents are useful in the treatment of clinical conditions that are either androgen-responsive or associated with androgen excess, such as acne. Effective anti-androgen therapy can be directed toward any of the regulatory steps in androgen production or action.

Anti-androgen therapy or androgen suppression can be achieved by many methods; however, some mandate surgery such as removal of the testis and orchiectomy. Anti-androgenic agents can also suppress androgen production in other ways, such as by inhibition of testicular steroidogenesis at the pituitary level, by inhibition of either luteinizing hormone-releasing hormone (LHRH) analogs or estrogens, by inhibition of testicular steroidogenesis at the testicular level using enzyme inhibitors, and by inhibition of androgen action by androgen receptor antagonists.

Anti-androgenic agents work by several mechanisms. There are those drugs which inhibit pituitary lutenizing hormone (LH) secretion and decrease testosterone production, termed “LHRH agonists,” and include, for example, nafarelin, leuprolide, goserelin, and buserelin. There are additional drugs which inhibit pituitary LH secretion and decrease testosterone production, but also inhibit androgen receptors, such as cyproterone acetate, zanoterone, and the progestins, like megestrol acetate, hydroxy-progesterone caproate and medrogestone. Other anti-androgenic drugs include the nonsteroidal agents hydroxyflutamide, Casodex®, nilutamide, and 5-alpha-reductase inhibitors (e.g., finasteride).

However, the prior art anti-androgenic agents are associated with a wide range of side effects upon systemic adminsitration, including impotence, loss of libido, gynecomastia, heat intolerance, and hot flashes among others. Some drugs have even been associated with fatal hepatotoxicity (see, e.g., D. K. Wysowski et al., Ann. Int. Med. 118(11):860-864 (1993)). Additionally, the prior art agents tend to have a very short half-life, necessitating more frequent and/or higher dosages (see for example, U.S. Pat. No. 4,150,127 to Anner et al., U.S. Pat. No. 4,412,993 to Sokolowski, and U.S. Pat. No. 4,673,673 to Laurent et al., U.S. Pat. No. 6,147,214 to Poli et al., U.S. Pat. No. 6,113,926 to Soler et al., U.S. Pat. No. 6,083,940 to Tanabe et al., all herein incorporated by reference).

In contrast, the present invention utilizes anti-androgenic agents at relatively low doses because of the local application of the composition thereby avoiding the undesirable side effects associated with the prior art.

In summary, there is a need for cosmetic products which effectively reduce skin oiliness and do so without major side effects. The method and compositions of the present invention beneficially provide a gentle yet effective means of sebum reduction by the topical application to the skin of compositions containing a carrier, surfactants, chylomicron disrupters, skin penetration enhancers, and anti-androgenic compounds.

### SUMMARY OF THE INVENTION

Sebum reduction methods and topical compositions for minimizing sebum on skin are disclosed. The present inventive method comprises applying to the skin a composition containing a carrier or mixtures thereof, a surfactant or mixtures thereof, a chylomicron disrupter or mixtures thereof, a skin penetration enhancer or mixtures thereof, and an anti-androgenic compound or mixtures thereof such that sebum production is minimized.

The carriers ethanol, acetone, and polyethylene glycol 400 are presently preferred, particularly as a mixture, in a quantities of about 70 to about 90 percent by weight ethanol, about 2 to about 10 percent by weight acetone, and about 2 to about 20 percent by weight polyethylene glycol 400 of the total composition.

Members of the polyoxyethylene group surfactants are preferably present in the composition. In a more preferred embodiment, the surfactants, HO(CH₂CH₂O)₇(CH₃CHCH₂O)₅₄(CH₂CH₂O)₇H and HO(CH₂CH₂O)₆(CH₃CHCH₂O)₃₉(CH₂CH₂O)₆H, are used in quantities of about 0.1 to about 2 percent by weight HO(CH₂CH₂O)₇(CH₃CHCH₂O)₅₄(CH₂CH₂O)₇H and about 0.1 to about 2 percent of HO(CH₂CH₂O)₆(CH₃CHCH₂O)₃₉(CH₂CH₂O)₆H of the total weight of the composition.

Anti-androgenic compounds, particularly saw palmetto extract and nettle extract, are presently preferred. In a more preferred embodiment, the quantities of anti-androgenic compounds present in the composition are about 1 to about 20 percent by weight saw palmetto extract, and about 1 to about 20 percent by weight of nettle extract.

In a preferred embodiment, the method entails applying to the skin a composition is comprised of a mixture containing about 70 to about 90 percent by weight ethanol, about 2 to about 10 percent by weight acetone, about 2 to about 20 percent by weight polyethylene glycol, about 0.1 to about 2 percent by weight surfactants HO(CH₂CH₂O)₇(CH₃CHCH₂O)₅₄(CH₂CH₂O)₇H, about 0.1 to about 2 percent by weight HO(CH₂CH₂O)₆(CH₃CHCH₂O)₃₉(CH₂CH₂O)₆H, about 0.5 to about 10 percent by weight urea, about 1 to about 20 percent by weight saw palmetto extract and about 1 to about 20 percent by weight nettle extract of the composition.

In yet another embodiment, the method comprises applying to skin a composition containing a polyoxyethylene surfactant or mixtures thereof and an anti-androgenic compound or mixtures thereof.

In a particularly preferred embodiment, the method comprises applying to skin a composition containing about 0.1 to about 2 percent by weight of surfactant HO(CH₂CH₂O)₇(CH₃CHCH₂O)₅₄(CH₂CH₂O)₇H and about 1 to about 20 percent by weight of saw palmetto extract.Another embodiment provides a method for reducing sebum secretion in mammals by applying to skin a composition containing about 0.1 to about 2 percent by weight of surfactant HO(CH₂CH₂O)₆(CH₃CHCH₂O)₃₉(CH₂CH₂O)₆H and about 1 to about 20 percent by weight of nettle extract.

This invention provides novel methods and compositions for effectively reducing sebum on the skin surface which do not have the deleterious side effects associated with the prior art, such as skin irritation, increased skin sensitivity, toxicity, scarring, or hypervitaminosis.

### DETAILED DESCRIPTION OF THE INVENTION

The present invention is directed to methods and improved compositions for reducing sebum on the surface of skin. Minimization of sebum is achieved by applying to the skin a composition containing a group of organic solvents, termed the carrier, a polyoxyethylene surfactant or mixtures thereof known to disrupt fat structure, a chylomicron disrupter or mixtures thereof which can disrupt fatty particles, a skin penetration enhancer or mixtures thereof, and an anti-androgenic compound or mixtures thereof which are known to modify the production of androgens and thereby sebum in humans.

The term “chylomicron disrupter” as used herein includes lipase inhibitors such as surfactants and other compounds that inhibit the secretion of chylomicrons.

In the present invention, the carrier is useful in dissolving the sebum on the skin surface and in deeper tissue as well. Presently preferred carriers contain alcohols, glycols, ketones, or mixtures thereof. More preferably, the carrier is a member of the group of ethanol, acetone, and polyethylene glycol 400 or mixtures thereof. The most preferred embodiment of the present invention contains a mixture of about 70 to about 90 percent by weight ethanol, about 2 to about 10 percent by weight acetone, and about 2 to about 20 percent by weight of polyethylene glycol 400.

The present invention also utilizes surfactants to enhance skin penetration. Preferably, polyoxyethylene surfactants are included in the inventive composition such as surfactant HO(CH₂CH₂O)₇(CH₃CHCH₂O)₅₄(CH₂CH₂O)₇H and surfactant HO(CH₂CH₂O)₆(CH₃CHCH₂O)₃₉(CH₂CH₂O)₆H being more preferred. Besides facilitating skin penetration, these two surfactants are known to inhibit the formation of monogylcerides which leads to problems in sebum accumulation on the skin. Most preferably, a mixture of HO(CH₂CH₂O)₇(CH₃CHCH₂O)₅₄(CH₂CH₂O)₇H in an amount of about 0.1 to about 2 percent by weight and HO(CH₂CH₂O)₆(CH₃CHCH₂O)₃₉(CH₂CH₂O)₆H in an amount of about 0.1 to about 2 percent by weight is present in the composition.

Chylomicron disrupters are another component of the present invention. A chylomicron is a spherical particle having a core of triglycerides surrounded by a monolayer of phospholipids, cholesterol, and apolipoproteins. Preferably the chylomicron disrupter is a member of the group consisting of orlistat, esterastin, 3,5-hydroxy-2-hexadeca-7,10-dienoic 1,3-lactone, 3,5-di-hydroxy-2-hexylhexadecanoic 1,3-lactonebitors, tetrahydroesterastin, 3,5-dihydroxy-2-hexylhexadeca-7,10-dienoic 1,3-lactone, 3,5-di-hydroxy-2-hexylhexadecanoic 1,3-lactone, (2S,3S,5S)-5-[(S)-2-formamido-4-methyl-valeryloxy]-2-hexyl-3-hydroxy-hexadecanoic 1,3-acid lactone, (2S,3S,5S,7Z,10Z)-5-[(S)-2-formamido-4-methyl-valeryloxy]-2-hexyl-3-hydroxy-7,10-hexadecadienoic 1,3 acid lactone, 1-(trans-4-isobutylcyclohexyl)-2-(phenylsulfonyloxy) ethanone, 4-methylpiperidine-1-carboxylic acid 4-phenoxyphenyl ester, N-[3-chloro-4-(trifluoromethyl)phenyl-N′-[3-(trifluoromethyl)phenyl]urea, N-formyl-L-valine-(S)-1-[[(2S,3S)-3-hexyl-4-oxo-2-oxetanyl]methyl]hexyl ester, (2S,3S,5S,7Z,10Z)-5-[(S)-2-acetamido-3-carbamoylpropionyloxy]-2-hexyl-3-hydroxy-7,10-hexadecadienoic lactone, (3S,4S)-4-[(1S,5R,7S,8R,9R,E)-8-hydroxy-1,3,5,7,9-pentamethyl-6-oxo-3-unde cenyl]-3-methyl-2-oxetanone, (3S,4S)-3-ethyl-4-[(1S,5R,7S,8R,9R,E)-8-hydroxy-1,3,5,7,9-pentamethyl-6-oxo-3-undecenyl]-2-oxetanone, 1,6-di(O-(carbamoyl)cyclohexanone oxime)hexane, and polyoxypropylene surfactants. Most preferably, a mixture of HO(CH₂CH₂O)₇(CH₃CHCH₂O)₅₄(CH₂CH₂O)₇H in an amount of about 0.1 to about 2 percent by weight and HO(CH₂CH₂O)₆(CH₃CHCH₂O)₃₉(CH₂CH₂O)₆H in an amount of about 0.1 to about 2 percent by weight is present in the composition.

Another component of the present invention is the skin penetration enhancer. Preferably, the skin penetration enhancer is a member of the group consisting of a water-dispersible acid polymer, a physiologically acceptable water soluble polar compound, and a substantially water-insoluble transdermal penetration enhancing compound.

More preferably, the skin penetration enhancer is a member of the group consisting of C4 to C16 aliphatic group substituted acetals, hemi-acetals, morpholines, alcohols, glycols, lactams, urea, cycloethylene urea, 1,3-dioxolone, 2-methyl-1-3-dioxolone, 1,3-dioxane, 2-methyl-1,3-dioxane, morpholine, N-methylmorpholine, N-dimethylformamide, dimethylsulfoxide, methylacetate, ethyllactate, monosaccharides, polysaccharides, amino acids, amino alcohols, diethylamine, cycloethylene carbonate, dioxolane, formamide, carbonate, glucose, urea, lactim, 1-dodecylazacycloheptan-2-one hexamethylenelauramide, N-methyl-2-pyrrolidone, a sucrose aliphatic acid ester, and nonionic surfactants.

It is presently preferred that the skin penetration enhancer is present in the composition in an amount of about 0.5 to about 10 percent by weight.

The present invention also utilizes anti-androgenic compounds as part of the composition. Preferably, the anti-androgenic compound is selected from the group consisting of saw palmetto extract, nettle herbs extract, willow herbs extract, terazosin, doxazosin, prazosin, tamsulosin 4-(3-(4-cyano-3-trifluoromethyl-phenyl)-5,5-dimethyl-2,4-dioxo-1-imidazoli dinyl)-butyl, isopropyl carbonate, 4-(4,4-dimethyl-2,5-dioxo-3-(4-nitrooxybutyl)-1-imidazolidinyl-2-trifluoro methyl-benzonitrile, and cyproterone acetate. Preferably, the anti-androgenic compound or mixture thereof is present in the composition in an amount of about 1 to about 40 percent by weight of the composition

More preferably, the anti-androgenic compound is a mixture of nettle extract and saw palmetto extract. Most preferably, the anti-androgenic compound is comprised of a mixture of about 1 to about 20 percent by weight saw palmetto extract and about 1 to about 20 percent by weight nettle extract.

The most preferable embodiment of the invention comprises a composition containing a mixture of about 70 to about 90 percent by weight ethanol, about 2 to about 10 percent by weight acetone, about 2 to about 20 percent by weight polyethylene glycol, about 0.1 to about 2 percent by weight HO(CH₂CH₂O)₇(CH₃CHCH₂O)₅₄(CH₂CH₂O)₇H, about 0.1 to about 2 percent by weight HO(CH₂CH₂O)₆(CH₃CHCH₂O)₃₉(CH₂CH₂O)₆H, about 0.5 to about 10 percent by weight urea, about 1 to about 20 percent by weight saw palmetto extract and about 1 to about 20 percent by weight nettle extract of the composition.

Optionally, the methods and compositions of the present invention may contain a fragrance or mixture thereof, a preservative, a stabilizer, a colorant, an opacifier, or an antioxidant. The most preferred fragrance is vanilla.

The composition can be supplied in solid or liquid form and can be applied with the hands or any convenient applicator.

### EXAMPLE 1

Method of Reducing Sebum on Skin by Application of Topical Composition

A topical composition was made according to the following formula to yield 1 kg of product:

| | |
|---|---|
| Ethanol, USP | 760 G |
| Acetone | 60 G |
| Polyethylene Glycol 400 | 120 G |
| HO(CH₂CH₂O)₇(CH₃CHCH₂O)₅₄(CH₂CH₂O)₇H | 10 G |
| HO(CH₂CH₂O)₆(CH₃CHCH₂O)₃₉(CH₂CH₂O)₆H | 10 G |
| Urea NF | 20 G |
| Vanillin Extract | 10 G |
| Saw Palmetto hydroalcoholic extract | 10 G |
| Nettle hydroalcoholic extract | 10 G |

The experiments were carried out on 12 volunteers of average age 23 years who had oily skin (lipid baseline of over 150 as measured by Sebumeter, see below). Two treatment sites were selected: forehead and nose. Before the experiment began, no cosmetics were used for a period of three days. On the day of the treatment, subjects were randomly assigned to either treatment or control group. The selected treatment sites were treated in the control group by applying water to the site in an amount of about 2-5 mL, followed by washing with soap (Neutrogena®) and water thoroughly, then drying using first cotton wool and then muslin cloth five minutes later. The treatment group applied the inventive composition (about 2-5 mL), gently rubbing into skin for about one minute. The inventive composition was left to dry on the skin for five minutes, then the preparation was reapplied to the sites using strong circular motion then washed with soap and water as in the control group. The quantities of lipids at the cutaneous surface of the forehead and nose were measured by the Sebumeter described below every 2 hours over a period of 8 hours to observe sebum secretion.

The testing unit used was a Sebumeter SM810®, which is commercially available from Courage and Khazaka GmbH and is the standard recognized instrument for sebum production measurement. The Sebumeter measures lipid on the skin via photometry of a special plastic strip which becomes transparent when it absorbs lipids. The plastic strip is extended over a mirror, which is connected to a spring. The measuring head of the device (comprised of spring, mirror and plastic strip) is pressed against the skin for 30 seconds. The value (g/cm²) is indicative of the amount of lipid on the skin. The measuring method is insensitive to humidity. Sebumeter readings (generally 3) are taken along the length of the area monitored and the Lipid Deposition Value, LDV, (g/cm²) is defined as the mean of the 3 readings. The Sebumeter plastic strip also detects natural skin lipids. The Sebumeter like other surface extraction measurements may not measure the entire lipid. If the skin topography is undulating it is possible that deposited lipid may not be extracted by the Sebumeter strip. The Sebumeter tape becomes saturated at a LDV of above about 300 g/cm². The rate of sebum secretion is measured as g/cm²/hr.

The results, expressed in lipid indices, are shown below.

TABLE I
Lipid Deposition Value (LDV) on the forehead in six treatment
subjects and six control subjects.

| | LDV Control, | LDV Treatment, |
|---|---|---|
| Time, | Mean ± SD | Mean ± SD |
| hr | (g/cm²) | (g/cm²) |
| 0 | 7.2 ± 3.2 | 3.2 ± 2.8 |
| 2 | 123.4 ± 56.4 | 35.8 ± 12.7 |
| 4 | 176.5 ± 34.3 | 42.1 ± 13.5 |
| 6 | 210.5 ± 19.6 | 56.8 ± 22.8 |
| 8 | 230.7 ± 34.5 | 68.9 ± 18.9 |

The difference between the LDV obtained with the untreated reference zone and the treated zone was significant at 2 hours, and was maintained at 4 hours, 6 hours and 8 hours. It is clear from these observations that the application of the composition significantly reduces the skin re-oiling after cleansing. Statistical significance was reached within the first two hours.

TABLE II
Lipid Deposition Value on the nose in six treatment subjects and six
control subjects.

| Time, | Control, Mean ± SD | Treatment, Mean ± SD |
|---|---|---|
| hr | (g/cm²) | (g/cm²) |
| 0 | 8.4 ± 4.6 | 6.2 ± 4.6 |
| 2 | 129.5 ± 44.4 | 48.6 ± 22.3 |
| 4 | 181.5 ± 38.6 | 62.7 ± 22.8 |
| 6 | 205.6 ± 22.8 | 74.3 ± 19.6 |
| 8 | 222.6 ± 27.9 | 77.9 ± 21.4 |

The difference between the LDV obtained with the untreated reference zone and the treated zone was significant at 2 hours, and was maintained at 4 hours, 6 hours and 8 hours. It is clear from these observations that the application of the composition in accordance with the invention significantly reduces the skin re-oiling after cleansing. Statistical significance was reached within the first two hours.

These results indicate that the method and composition of the present invention significantly reduce the secretion of sebum and its removal from the skin. Additional studies lasting for six weeks showed that application for two or three times per day for one week substantially lowered the sebum secretion for up to three weeks.

TABLE III
Daily measurements of lipids on the forehead in multiple daily use
of composition

| Time, | Mean ± SD |
|---|---|
| day | (g/cm²) |
| 7 | 78.4 ± 24.6 |
| 14 | 85.6 ± 44.4 |
| 21 | 91.5 ± 38.6 |
| 28 | 143.6 ± 42.8 |
| 35 | 210.5 ± 29.8 |

The measurements were made on six volunteers as described above. Once a day measurements were made in the morning.

### EXAMPLE 2

Topical Gel

A gel was prepared having the following composition:

| Ingredient | Percent by Weight |
|---|---|
| HO(CH₂CH₂O)₆(CH₃CHCH₂O)₃₉(CH₂CH₂O)₆H | 1.00 |
| HO(CH₂CH₂O)₇(CH₃CHCH₂O)₅₄(CH₂CH₂O)₇H | 1.00 |
| Urea | 2.00 |
| Vanillin | 1.00 |
| Saw Palmetto extract | 1.00 |
| Nettle extract | 1.00 |
| Propylene glycol | 19.00 |
| Ethanol | 19.00 |
| Carboxyvinyl polymer [Carbomer 940 ®] | 1.00 |
| Hydroxyethyl cellulose | 0.40 |
| Benzyl alcohol | 1.00 |
| Sodium hydroxide 1N | to pH 6 |
| Distilled water | balance |

The components other than sodium hydroxide were combined to yield a homogeneous dispersion. Addition of sodium hydroxide caused the mixture to gel yielding a ready-to-use semisolid.

### EXAMPLE 3

Topical Cream

A cream was prepared consisting of:

| Ingredient | Percent by Weight |
|---|---|
| HO(CH₂CH₂O)₇(CH₃CHCH₂O)₅₄(CH₂CH₂O)₇H | 1.00 |
| HO(CH₂CH₂O)₆(CH₃CHCH₂O)₃₉(CH₂CH₂O)₆H | 1.00 |
| Urea | 2.00 |
| Vanillin | 1.00 |
| Saw Palmetto extract | 1.00 |
| Nettle extract | 1.00 |
| Stearic acid | 7.00 |
| Stearyl alcohol | 5.00 |
| Cetyl alcohol | 2.00 |
| Glycerin | 10.00 |
| Sodium laurylsulfate | 1.00 |
| Propylparaben | 0.05 |
| Methylparaben | 0.25 |
| Disodium edetate | 0.05 |
| Distilled water | balance |

The first nine ingredients were mixed then heated to approximately 70° C. to produce a uniform melt. The remaining ingredients were combined, then heated to approximately 75° C., then added with mixing to the previously prepared melt. The emulsion thus formed was subsequently homogenized then cooled to yield a smooth white cream.

### EXAMPLE 4

Topical Lotion

A lotion was prepared having the following composition:

| Ingredient | Percent by Weight |
|---|---|
| HO(CH₂CH₂O)₆(CH₃CHCH₂O)₃₉(CH₂CH₂O)₆H | 1.00 |
| HO(CH₂CH₂O)₇(CH₃CHCH₂O)₅₄(CH₂CH₂O)₇H | 1.00 |
| Urea | 2.00 |
| Vanillin | 1.00 |
| Saw Palmetto extract | 1.00 |
| Nettle extract | 1.00 |
| Glyceryl monostearate | 1.00 |
| Isopropyl palmitate | 4.00 |
| Polyethylene glycol 400 | 2.00 |
| Glycerin | 10.00 |
| Methylparaben | 0.10 |
| Sodium cetylsulfate | 5.00 |
| Distilled water | balance |

The first nine ingredients were combined and heated to approximately 70.degrees C. then added with agitation to a mixture of the remaining ingredients which were also heated to about 70.degrees C. The resulting emulsion mixture was homogenized and cooled to produce a smooth, white, pourable lotion.

The topical formulations presented herein are examples of typical gel, cream, lotion, or solution dosage forms of active compounds for use in the treatment of sebaceous oil production. Example 1 is intended for immediate cleansing and repeated use. Other examples are primarily intended for chronic use. Other optional components can be added or ratios of ingredients can be adjusted to enhance cosmetic acceptability of the formulations. Additionally, these alterations can be made to customize the composition toward a particular active compound, for example to ensure solubilization or to enhance chemical or physical stability. Optional components would include viscosity adjusters such as celluloses, emollient oils such as mineral oil or glycerides, humectants such as polyols, cosolvents such as isopropyl alcohol or acetone, emulsifying agents of the anionic, cationic and nonionic types, preservatives, antioxidants, opacifiers, colorants,and perfumes.

From the foregoing, it will be observed that numerous modifications and variations can be effected without departing from the true spirit and scope of the present invention. It is to be understood that no limitation with respect to the specific examples presented is intended or should be inferred. The disclosure is intended to cover by the appended claims modifications as fall within the scope of the claims.

We claim:

1. A method for reducing sebum secretion in mammals by applying to skin a topical composition containing a carrier or mixtures thereof, a surfactant or mixtures thereof, a chylomicron disrupter or mixtures thereof, a skin penetration enhancer or mixtures thereof, and an anti-androgenic compound or mixtures thereof.

2. The method of claim 1 wherein the carrier is selected from the group consisting of alcohols, ketones, and glycols.

3. The method of claim 1 wherein the carrier is a mixture comprising ethanol, acetone, and polyethylene glycol 400.

4. The method of claim 1 wherein the surfactant is a polyoxypropylene surfactant.

5. The method of claim 1 wherein the surfactant is selected from the group consisting of HO(CH₂CH₂O)₇(CH₃CHCH₂O)₅₄(CH₂CH₂O)₇H and HO(CH₂CH₂O)₆(CH₃CHCH₂O)₃₉(CH₂CH₂O)₆H.

6. The method of claim 1 wherein the chylomicron disrupter is selected from the group consisting of orlistat, esterastin, 3,5-hydroxy-2-hexadeca-7,10-dienoic 1,3-lactone, 3,5-di-hydroxy-2-hexylhexadecanoic 1,3-lactonebitors, tetrahydroesterastin, 3,5-dihydroxy-2-hexylhexadeca-7,10-dienoic 1,3-lactone, 3,5-di-hydroxy-2-hexylhexadecanoic 1,3-lactone, (2S,3S,5S)-5-[(S)-2-formamido-4-methyl-valeryloxy]-2-hexyl-3-hydroxy-hexadecanoic 1,3 acid lactone, (2S,3S,5S,7Z,10Z)-5-[(S)-2-formamido-4-methyl-valeryloxy]-2-hexyl-3-hydroxy-7,10-hexadecadienoic 1,3 acid lactone, 1-(trans-4-isobutylcyclohexyl)-2-(phenylsulfonyloxy) ethanone, 4-methylpiperidine-1-carboxylic acid 4-phenoxyphenyl ester, N-[3-chloro-4-(trifluoromethyl)phenyl-N′-[3-(trifluoromethyl)phenyl]urea, N-formyl-L-valine-(S)-1-[[(2S,3S)-3-hexyl-4-oxo-2-oxetanyl]methyl]hexyl ester, (2S,3S,5S,7Z,10Z)-5-[(S)-2-acetamido-3-carbamoylpropionyloxy]-2-hexyl-3-hydroxy-7,10-hexadecadienoic lactone, (3S,4S)-4-[(1S,5R,7S,8R,9R,E)-8-hydroxy-1,3,5,7,9-pentamethyl-6-oxo-3-unde cenyl]-3-methyl-2-oxetanone, (3S,4S)-3-ethyl-4-[(1S,5R,7S,8R,9R,E)-8-hydroxy-1,3,5,7,9-pentamethyl-6-oxo-3-undecenyl]-2-oxetanone, 1,6-di(O-(carbamoyl)cyclohexanone oxime)hexane, and polyoxypropylene surfactants.

7. A method for reducing sebum secretion in mammals by applying to skin a topical composition containing terazosin.

8. The method of claim 1 wherein the skin penetration enhancer is selected from the group consisting of a water-dispersible acid polymer, a physiologically acceptable water soluble polar compound, and a substantially water-insoluble transdermal penetration enhancing compound.

9. The method of claim 1 wherein the skin penetration enhancer is selected from the group consisting of C4 to C16 aliphatic group substituted acetals, hemi-acetals, morpholines, alcohols, glycols, lactams, urea, cycloethylene urea, 1,3-dioxolone, 2-methyl-1-3-dioxolone, 1,3-dioxane, 2-methyl-1,3-dioxane, morpholine, N-methylmorpholine, N-dimethylformamide, dimethylsulfoxide, methylacetate, ethyllactate, monosaccharides, polysaccharides, amino acids, amino alcohols, diethylamine, cycloethylene carbonate, dioxolane, formamide, carbonate, glucose, urea, lactim, 1-dodecylazacycloheptan-2-one hexamethylenelauramide, N-methyl-2-pyrrolidone, a sucrose aliphatic acid ester, and nonionic surfactants.

10. The method of claim 1 wherein the anti-androgenic compound is selected from the group consisting of saw palmetto, nettle herbs, willow herbs, terazosin, doxazosin, prazosin, tamsulosin 4-(3-(4-cyano-3-trifluoromethyl-phenyl)-5,5-dimethyl-2,4-dioxo-1-imidazoli dinyl)-butyl, isopropyl carbonate, 4-(4,4-dimethyl-2,5-dioxo-3-(4-nitrooxybutyl)-1-imidazolidinyl-2-trifluoro methyl-benzonitrile, and cyproterone acetate.

11. The method of claim 1 wherein the composition further comprises a component selected from the group consisting of a viscosity adjuster, emollient oil, humectant, emulisifying agent, fragrance, preservative, opacifier, and a stabilizer.

12. The method of claim 1 wherein the composition is comprised of a mixture containing about 19 percent by weight ethanol, about 19 percent by weight carboxyvinyl polymer, about 1 percent by weight hydroxyethyl cellulose, about 1 percent by weight benzyl alcohol, about 19 percent by weight propylene glycol, about 1 percent by weight HO(CH₂CH₂O)₇(CH₃CHCH₂O)₅₄(CH₂CH₂O)₇H, about 1 percent by weight HO(CH₂CH₂O)₆(CH₃CHCH₂O)₃₉(CH₂CH₂O)₆H, about 2 percent by weight urea, about 1 percent by weight saw palmetto extract and about 1 percent by weight nettle extract of the composition and is about pH 6.

13. The method of claim 1 wherein the composition is comprised of a mixture containing about 5 percent by weight stearyl alcohol, about 2 percent by weight cetyl alcohol, about 1 percent by weight sodium laurylsulfate, about 0.05 percent by weight propylparaben, about 0.25 percent by weight methylparaben, about 0.05 percent disodium edatate, about 1 percent by weight vanillin, about 7 percent by weight stearic acid, about 10 percent by weight glycerin, about 1 percent by weight HO(CH₂CH₂O)₇(CH₃CHCH₂O)₅₄(CH₂CH₂O)₇H, about 1 percent by weight HO(CH₂CH₂O)₆(CH₃CHCH₂O)₃₉(CH₂CH₂O)₆H, about 2 percent by weight urea, about 1 percent by weight saw palmetto extract and about 1 percent by weight nettle extract of the composition.

14. The method of claim 1 wherein the composition is comprised of a mixture containing about 1 percent by weight glyceryl monostearate, about 4 percent by weight isopropyl palmitate, about 2 percent by weight polyethylene glycol 400, about 10 percent by weight glycerin, about 1 percent by weight vanillin, about 0.1 percent by weight methylparaben, about 5 percent by weight sodium cetylsulfate, about 1 percent by weight HO(CH₂CH₂O)₇(CH₃CHCH₂O)₅₄(CH₂CH₂O)₇H, about 1 percent by weight HO(CH₂CH₂O)₆(CH₃CHCH₂O)₃₉(CH₂CH₂O)₆H, about 2 percent by weight urea, about 1 percent by weight saw palmetto extract and about 1 percent by weight nettle extract of the composition.

15. The method of claim 1 wherein the composition is in the form of a liquid.

16. The method of claim 1 wherein the composition is in the form of a solid.

17. The method of claim 1 wherein the carrier is present in the composition in an amount of about 2 to about 90 percent by weight of the composition.

18. The method of claim 1 wherein the carrier is comprised of a mixture of about 70 to about 90 percent by weight ethanol, about 2 to about 10 percent by weight acetone, and about 2 to about 20 percent by weight polyethylene glycol.

19. The method of claim 1 wherein the surfactant is present in the composition in an amount of about 0.1 to about 2 percent by weight of the composition.

20. The method of claim 1 wherein the surfactant is comprised of a mixture of about 0.1 to about 2 percent by weight HO(CH₂CH₂O)₇(CH₃CHCH₂O)₅₄(CH₂CH₂O)₇H and about 0.1 to about 2 percent by weight HO(CH₂CH₂O)₆(CH₃CHCH₂O)₃₉(CH₂CH₂O)₆H.

21. The method of claim 1 wherein the composition further comprises an antioxidant.

22. The method of claim 1 wherein the composition further comprises a colorant.

23. The method of claim 1 wherein the skin penetration enhancer is present in the composition in an amount of about 0.5 to about 10 percent by weight of the composition.

24. The method of claim 1 wherein the anti-androgenic compound or mixture thereof is present in the composition in an amount of about 1 to about 40 percent by weight of the composition.

25. The method of claim 1 wherein the anti-androgenic compound is comprised of a mixture of about 1 to about 20 percent by weight saw palmetto extract and about 1 to about 20 percent by weight nettle extract.

26. The method of claim 1 wherein the composition is comprised of a mixture containing about 70 to about 90 percent by weight ethanol, about 2 to about 10 percent by weight acetone, about 2 to about 20 percent by weight polyethylene glycol, about 0.1 to about 2 percent by weight HO(CH₂CH₂O)₇(CH₃CHCH₂O)₅₄(CH₂CH₂O)₇H, about 0.1 to about 2 percent by weight HO(CH₂CH₂O)₆(CH₃CHCH₂O)₃₉(CH₂CH₂O)₆H, about 0.5 to about 10 percent by weight urea, about 1 to about 20 percent by weight saw palmetto extract and about 1 to about 20 percent by weight nettle extract of the composition.

27. A method for reducing sebum secretion in mammals by applying to skin a composition containing a polyoxyethylene surfactant or mixtures thereof and an anti-androgenic compound or mixtures thereof.

28. A method for reducing sebum secretion in mammals by applying to skin a composition containing about 0.1 to about 2 percent by weight of HO(CH₂CH₂O)₇(CH₃CHCH₂O)₅₄(CH₂CH₂O)₇H and about 1 to about 20 percent by weight of saw palmetto extract.

29. A method for reducing sebum secretion in mammals by applying to skin a composition containing about 0.1 to about 2 percent by weight of HO(CH₂CH₂O)₆(CH₃CHCH₂O)₃₉(CH₂CH₂O)₆H and about 1 to about 20 percent by weight of nettle extract.

30. A topical composition for reducing sebum secretion in mammals containing a carrier or mixtures thereof, a surfactant or mixtures thereof, a chylomicron disrupter or mixtures thereof, a skin penetration enhancer or mixtures thereof, and an anti-androgenic compound or mixtures thereof.

31. The composition of claim 30 wherein the carrier is selected from the group consisting of alcohols, ketones, and glycols.

32. The composition of claim 30 wherein the carrier is a mixture comprising ethanol, acetone, and polyethylene glycol 400.

33. The composition of claim 30 wherein the surfactant is a polyoxypropylene surfactant.

34. The composition of claim 30 wherein the surfactant is selected from the group consisting of HO(CH₂CH₂O)₇(CH₃CHCH₂O)₅₄(CH₂CH₂O)₇H and HO(CH₂CH₂O)₆(CH₃CHCH₂O)₃₉(CH₂CH₂O)₆H.

35. The composition of claim 30 wherein the chylomicron disrupter is selected from the group consisting of orlistat, esterastin, 3,5-hydroxy-2-hexadeca-7,10-dienoic 1,3-lactone, 3,5-di-hydroxy-2-hexylhexadecanoic 1,3-lactonebitors, tetrahydroesterastin, 3,5-dihydroxy-2-hexylhexadeca-7,10-dienoic 1,3-lactone, 3,5-di-hydroxy-2-hexylhexadecanoic 1,3-lactone, orlistat, (2S,3S,5S)-5-[(S)-2-formamido-4-methyl-valeryloxy]-2-hexyl-3-hydroxy-hexadecanoic 1,3 acid lactone, (2S,3S,5S,7Z,10Z)-5-[(S)-2-formamido-4-methyl-valeryloxy]-2-hexyl-3-hydroxy-7,10-hexadecadienoic 1,3 acid lactone, 1-(trans-4-isobutylcyclohexyl)-2-(phenylsulfonyloxy) ethanone, 4-methylpiperidine-1-carboxylic acid 4-phenoxyphenyl ester, N-[3-chloro-4-(trifluoromethyl)phenyl-N′-[3-(trifluoromethyl)phenyl]urea, N-formyl-L-valine-(S)-1-[[(2S,3S)-3-hexyl-4-oxo-2-oxetanyl]methyl]hexyl ester, (2S,3S, 5S,7Z,10Z)-5-[(S)-2-acetamido-3-carbamoylpropionyloxy]-2-hexyl-3-hydroxy-7,10-hexadecadienoic lactone, (3S,4S)-4-[(1S,5R,7S,8R,9R,E)-8-hydroxy-1,3,5,7,9-pentamethyl-6-oxo-3-unde cenyl]-3-methyl-2-oxetanone, (3S,4S)-3-ethyl-4-[(1S,5R,7S,8R,9R,E)-8-hydroxy-1,3,5,7,9-pentamethyl-6-oxo-3-undecenyl]-2-oxetanone, 1,6-di(O-(carbamoyl)cyclohexanone oxime)hexane, and polyoxypropylene surfactants.

36. A topical composition for reducing sebum secretion in mammals containing terazosin.

37. The composition of claim 30 wherein the skin penetration enhancer is selected from the group consisting of a water-dispersible acid polymer, a physiologically acceptable water soluble polar compound, and a substantially water-insoluble transdermal penetration enhancing compound.

38. The composition of claim 30 wherein the skin penetration enhancer is selected from the group consisting of C4 to C16 aliphatic group substituted acetals, hemi-acetals, morpholines, alcohols, glycols, lactams, urea, cycloethylene urea, 1,3-dioxolone, 2-methyl-1-3-dioxolone, 1,3-dioxane, 2-methyl-1,3-dioxane, morpholine, N-methylmorpholine, N-dimethylformamide, dimethylsulfoxide, methylacetate, ethyllactate, monosaccharides, polysaccharides, amino acids, amino alcohols, diethylamine, cycloethylene carbonate, dioxolane, formamide, carbonate, glucose, urea, lactim, 1-dodecylazacycloheptan-2-one hexamethylenelauramide, N-methyl-2-pyrrolidone, a sucrose aliphatic acid ester, and nonionic surfactants.

39. The composition of claim 30 wherein the anti-androgenic compound is selected from the group consisting of saw palmetto, nettle herbs, willow herbs, terazosin, doxazosin, prazosin, tamsulosin 4-(3-(4-cyano-3-trifluoromethyl-phenyl)-5,5-dimethyl-2,4-dioxo-1-imidazoli dinyl)-butyl, isopropyl carbonate, 4-(4,4-dimethyl-2,5-dioxo-3-(4-nitrooxybutyl)-1-imidazolidinyl-2-trifluoro methyl-benzonitrile, and cyproterone acetate.

40. The composition of claim 30 further comprising a component selected from the group consisting of a viscosity adjuster, emollient oil, humectant, emulsifying agent, fragrance, preservative, opacifier, and a stabilizer.

41. The composition of claim 30 wherein the composition is comprised of a mixture containing about 19 percent by weight ethanol, about 19 percent by weight carboxyvinyl polymer, about 1 percent by weight hydroxyethyl cellulose, about 1 percent by weight benzyl alcohol, about 19 percent by weight propylene glycol, about 1 percent by weight HO(CH₂CH₂O)₇(CH₃CHCH₂O)₅₄(CH₂CH₂O)₇H, about 1 percent by weight HO(CH₂CH₂O)₆(CH₃CHCH₂O)₃₉(CH₂CH₂O)₆H, about 2 percent by weight urea, about 1 percent by weight saw palmetto extract and about 1 percent by weight nettle extract of the composition and is about pH 6.

42. The composition of claim 30 wherein the composition is comprised of a mixture containing about 5 percent by weight stearyl alcohol, about 2 percent by weight cetyl alcohol, about 1 percent by weight sodium laurylsulfate, about 0.05 percent by weight propylparaben, about 0.25 percent by weight methylparaben, about 0.05 percent disodium edatate, about 1 percent by weight vanillin, about 7 percent by weight stearic acid, about 10 percent by weight glycerin, about 1 percent by weight HO(CH₂CH₂O)₇(CH₃CHCH₂O)₅₄(CH₂CH₂O)₇H, about 1 percent by weight HO(CH₂CH₂O)₆(CH₃CHCH₂O)₃₉(CH₂CH₂O)₆H, about 2 percent by weight urea, about 1 percent by weight saw palmetto extract and about 1 percent by weight nettle extract of the composition.

43. The composition of claim 30 wherein the composition is comprised of a mixture containing about 1 percent by weight glyceryl monostearate, about 4 percent by weight isopropyl palmitate, about 2 percent by weight polyethylene glycol 400, about 10 percent by weight glycerin, about 1 percent by weight vanillin, about 0.1 percent by weight methylparaben, about 5 percent by weight sodium cetylsulfate, about 1 percent by weight HO(CH₂CH₂O)₇(CH₃CHCH₂O)₅₄(CH₂CH₂O)₇H, about 1 percent by weight HO(CH₂CH₂O)₆(CH₃CHCH₂O)₃₉(CH₂CH₂O)₆H, about 2 percent by weight urea, about 1 percent by weight saw palmetto extract and about 1 percent by weight nettle extract of the composition.

44. The composition of claim 30 in the form of a liquid.

45. The composition of claim 30 in the form of a solid.

46. The composition of claim 30 wherein the carrier is present in the composition in an amount of about 2 to about 90 percent by weight of the composition.

47. The composition of claim 30 wherein the carrier is comprised of a mixture of about 70 to about 90 percent by weight ethanol, about 2 to about 10 percent by weight acetone, and about 2 to about 20 percent by weight polyethylene glycol.

48. The composition of claim 30 wherein the surfactant is present in the composition in an amount of about 0.1 to about 2 percent by weight of the composition.

49. The composition of claim 30 wherein the surfactant is comprised of a mixture of about 0.1 to about 2 percent by weight HO(CH₂CH₂O)₇(CH₃CHCH₂O)₅₄(CH₂CH₂O)₇H and about 0.1 to about 2 percent by weight HO(CH₂CH₂O)₆(CH₃CHCH₂O)₃₉(CH₂CH₂O)₆H.

50. The composition of claim 30 wherein the chylomicron disrupter is present in the composition in an amount of about 0.1 to about 2 percent by weight of the composition.

51. The composition of claim 30 wherein the composition further comprises an antioxidant.

52. The composition of claim 30 wherein the skin penetration enhancer is present in the composition in an amount of about 0.5 to about 10 percent by weight of the composition.

53. The composition of claim 30 wherein the anti-androgenic compound is present in the composition in an amount of about 1 to about 40 percent by weight of the composition.

54. The composition of claim 30 wherein the anti-androgenic compound is comprised of a mixture of about 1 to about 20 percent by weight saw palmetto extract and about 1 to about 20 percent by weight nettle extract.

55. The composition of claim 30 wherein the composition is comprised of a mixture containing about 70 to about 90 percent by weight ethanol, about 2 to about 10 percent by weight acetone, about 2 to about 20 percent by weight polyethylene glycol, about 0.1 to about 2 percent by weight HO(CH₂CH₂O)₆(CH₃CHCH₂O)₃₉(CH₂CH₂O)₆H, about 0.1 to about 2 percent by weight HO(CH₂CH₂O)₆(CH₃CHCH₂O)₃₉(CH₂CH₂O)₆H, about 0.5 to about 10 percent by weight of urea, about 1 to about 20 percent by weight saw palmetto extract and about 1 to about 20 percent by weight nettle extract of the composition.

56. A composition for reducing sebum secretion in mammals containing a polyoxyethylene surfactant or mixtures thereof and an anti-androgenic compound or mixtures thereof.

57. A composition for reducing sebum secretion in mammals containing about 0.1 to about 2 percent by weight of poloxamer 331 and about 1 to about 20 percent by weight of saw palmetto extract.

58. A composition for reducing sebum secretion in mammals containing about 0.1 to about 2 percent by weight of HO(CH₂CH₂O)₆(CH₃CHCH₂O)₃₉(CH₂CH₂O)₆H and about 1 to about 20 percent by weight of nettle extract.

59. A method for reducing sebum secretion in mammals by applying to skin a topical composition containing an anti-androgenic compound or mixtures thereof.

60. The method of claim 59 wherein the anti-androgenic compound is selected from the group consisting of terazosin, doxazosin, prazosin, tamsulosin.

61. A topical composition for reducing sebum secretion in mammals containing an anti-androgenic compound or mixtures thereof.

62. The composition of claim 61 wherein the anti-androgenic compound is selected from the group consisting of terazosin, doxazosin, prazosin, tamsulosin.
